(19) United States
(12) Patent Application Publication
(10) Pub. No.: US 2002/0123531 A1
(43) Pub. Date: Sep. 5, 2002

(54) Triaryl methane dyes and their use as photochemotherapeutic agents

(76) Inventors: Guilherme Luiz Indig, Madison, WI (US); Jeremy Bartlett, Middleton, WI (US)

Correspondence Address: DEWITT ROSS & STEVENS S.C., 8000 EXCELSIOR DR, SUITE 401, MADISON, WI, 53717-1914, US

(21) Appl. No.: 09/863,149

(22) Filed: May 22, 2001

Related U.S. Application Data
(63) Continuation-in-part of application No. 09/753,472, filed on Jan. 3, 2001.

(51) Int. Cl. A61K 31/135

(52) U.S. Cl. 514/648; 604/20

(57) ABSTRACT

Disclosed are compounds of the formula:

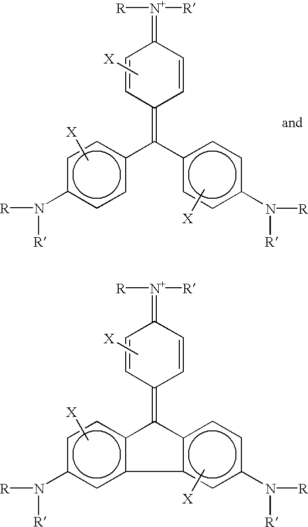

wherein R and R′ are hydrogen or C₁, to C₆ alkyl, and X is halo, pharmaceutical compositions containing the compounds as active ingredients, and use of the compounds in the photodynamic treatment of cancers and the purging of cancer cells from biological material to be transplanted into cancer patients.

This is a continuation-in-part of co-pending application Ser. No. 09/753,472, filed Jan. 3, 2001, the entire content of which is incorporated herein.

### FIELD OF THE INVENTION

The invention is directed to a method of treating cancer using triarylmethane dyes as photochemotherapeutic agents. The invention is also directed to a method of purging cancerous cells from non-cancerous cells in autologous bone marrow grafts.

### BIBLIOGRAPHIC CITATIONS

Complete bibliographic citations to the references discussed herein are contained in the Bibliography section, directly preceding the Claims.

### BACKGROUND AND DESCRIPTION OF THE RELATED ART

It is known that cancerous cells, such as tumor cells and leukemia cells, can be selectively purged from non-cancerous cells by photochemical methods. These methods are particularly useful in purging leukemia cells from a bone marrow graft before bone marrow transplantation. For instance, Merocyanine 540 (MC-540), a photosensitizing dye, has been used in photochemical purging of a patient's own (i.e., autologous) bone marrow graft. The effectiveness of MC-540-mediated photochemical purging, however, differs markedly in different leukemia cells lines.⁴⁶

Yamazaki and Sieber (1997)⁴⁵ found, however, that the selective lethality of MC-540 for leukemia cells could be synergistically increased by using MC-540 in conjunction with an alkyl-lysophospholipid, rac-2-methyl-1-octadecyl-glycero-(3)-phosphocholine (ET-18-OCH₃). These authors found that when photodynamic therapy (PDT) with MC-540 was followed by incubating the cells in ET-18-OCH₃, the MC-540-mediated photoinactivation of leukemia cells was synergistically enhanced, while the treatment only minimally reduced the survival of normal granulocyte-macrophage progenitors.

On the basis of a comprehensive investigation involving more than 200 cell lines/types of melanoma, adenocarcinoma, transitional cell carcinoma, squamous cell carcinoma, and normal epithelial cells, Chen has demonstrated that enhanced mitochondrial membrane potential is a prevalent cancer cell phenotype.¹ Only approximately 2% of all cells tested so far disobey this apparently dominant precept. Higher electric potentials have also been observed in the plasma membrane of a variety of carcinoma cells as compared to normal epithelial cells. Because cell and mitochondrial membrane potentials are negative inside, extensively conjugated cationic molecules displaying appropriate structural features can be electrophoretically driven through these membranes and accumulate into the cytosol and inside cell mitochondria. The mitochondrial membrane potential is typically more than 60 mV higher in carcinoma cells than in normal epithelial cells.^{1,2} As a result, a number of cationic dyes preferentially accumulate and are retained in a variety of tumor cells, presumably because the mitochondria of these cells are not capable of excreting the dyes with the same efficiency as normal cells.

The preferential uptake and retention of a variety of extensively conjugated cationic compounds by tumor cells have motivated the examination of mitochondrial targeting as a relevant therapeutic strategy for both chemotherapy and photochemotherapy of neoplastic diseases.³⁻⁷ However, the structural parameters that control the accumulation of these compounds into cell mitochondria are not entirely understood, and the lack of a robust model to describe the relationship between molecular structure and mitochondrial accumulation has prevented mitochondrial targeting from becoming a more dependable therapeutic strategy. Described herein is a method of treating cancer that utilizes cationic, triarylmethane dyes. While the invention is not limited to a particular mode of action, it is thought that the destruction of tumor cells wrought by the method arises via selective accumulation of the dye in the mitochondria of tumor cells.

Since 1953, when Nussenzweig⁸ first described the inactivation of the protozoan parasite Trypanosoma cruzi (the vector responsible for Chagas' disease) by the cationic triarylmethane dye crystal violet (CV⁺), this triarylmethane dye has been extensively used in blood banks in underdeveloped areas to prevent transfusion-associated transmission of Chagas' disease (American trypanosomiasis).⁹⁻¹⁴ CV⁺ does not cause severe side effects in patients who receive blood treated with it, nor are the functions of blood cells jeopardized as a result of the chemoprophylaxis.^{12,14} The safety of CV⁺ is further demonstrated by its use as an anthelmintic, an antiseptic in umbilical cords of newborns and burn patients, and a colorant in food and cosmetics.^{11,15,16} The trypanocidal activity of CV⁺ is known to develop at the mitochondrial level,¹⁰ and because it has been demonstrated that light enhances the trypanocidal effects of this triarylmethane (TAM⁺) dye,⁹⁻¹⁴ it was thought that CV⁺ might be a candidate for use in photodynamic therapies to kill cancer cells selectively, and/or to inhibit the growth, spread, and proliferation of cancer cells.

### SUMMARY OF THE INVENTION

A first embodiment of the invention is directed to a method of killing cancer cells or inhibiting growth of cancer cells, in vitro, in vivo, or ex vivo. The method comprises first contacting the cancer cells with a compound selected from the group consisting of:

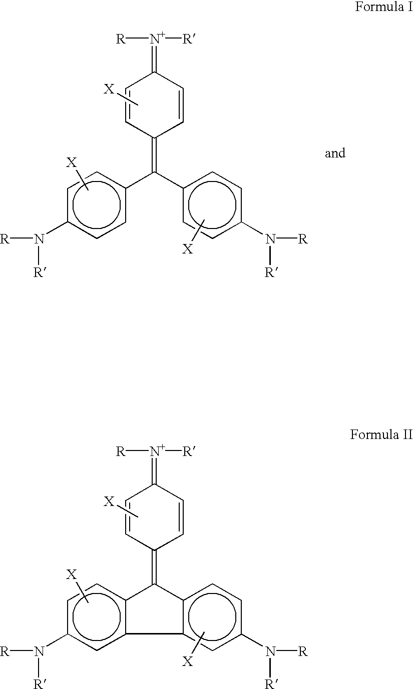

wherein each R and R′ is independently selected from the group consisting of hydrogen and C₁-C₆ linear or branched alkyl, and each X is independently selected from the group consisting of hydrogen and halo (chloro, flouro, bromo, iodo), and pharmaceutically-suitable salts thereof. In a preferred embodiment, R and R′ are not all simultaneously methyl. Then, the cancer cells so treated are exposed to radiation of a suitable wavelength to photoactivate the compound, whereby cancer cell death or cancer cell growth inhibition results. The method can be used to treat solid neoplastic tumors and circulating neoplasms such as leukemia and the like.

A second embodiment of the invention is directed to a method of purging malignant cells from a mixture containing malignant and non-magignant cells. The method comprises contacting the mixture with one or more compounds as described in the preceding paragraph. The mixture so treated is then exposed to radiation of a suitable wavelength to photoactivate the compound, thereby inducing death of malignant cells in the mixture.

This embodiment of the invention is preferred to be used in preparing autologous bone marrow transplants for reimplantation into the subject from which the transplant was taken. The subject will normally be a mammal (human or other mammal) suffering from a neoplasm involving the cells found in bone marrow, such as leukemia.

A third embodiment of the invention is drawn to pharmaceutical compositions for the photo-initiated treatment of neoplastic cell growth in mammals, including humans. The composition comprises an amount of one or more compounds as described hereinabove, in combination with a pharmaceutically-suitable diluent or carrier, the amount being effective to inhibit neoplastic cell growth upon being contacted with the neoplastic cells and then activated by exposure to radiation of a suitable wavelength (as described below).

A preferred compound for inclusion in the pharmaceutical composition is a compound of Formula I, wherein all of the X substituents are hydrogen, five of the six R and R′ groups are methyl, and the remaining R or R′ group is hydrogen. This compound has been given the trivial name methyl violet 2B (“MV2B”).

It has been found that the cationic triaryl methane dyes described herein (and referred to generally as “TAM⁺” dyes) exhibit pronounced and unexpected phototoxicity toward leukemia cells and low toxicity toward normal hematopoietic cells. On the basis of the selectivity with which the phototoxic effect of these compounds develops toward tumor cells as compared to normal cells, the principal advantage and benefit of the invention is that these triarylmethane dyes can be used in photodynamic therapy to destroy and/or inhibit the growth of cancer cells, while leaving non-cancerous cells viable. A primary use of the invention, therefore, is as a novel purging protocol to promote the elimination of residual tumor cells from autologous bone marrow grafts with minimum toxicity toward normal cells.

Additionally, many of the compounds described herein have absorbance maxima in the near infrared region. These compounds are well-suited for photodynamic therapy, especially in solid tumors, because near-infrared light penetrates tissues better than does visible light.

### BRIEF DESCRIPTION OF THE DRAWINGS

FIG. 1 shows the photoinactivation of L1210 leukemia cells (solid lines) and murine CFU-GM cells (broken lines) sensitized by TAM⁺ dyes. Data points represent mean colony counts ± standard errors of four replicate culture dishes. Untreated leukemia cells (0 min) generated a mean number of 134.5 colonies per 400 cells plated. Untreated bone marrow cells (0 min) generated a mean number of 482.5 granulocyte-macrophage colonies per 500,000 nucleated cells plated. Cells incubated for 60 minutes with TAM⁺ 1.0×10⁻⁶ M. Fluence rate=27 W/m².

FIG. 2 is a series of photographs illustrating the chromatographic separation of Crystal Violet and various demethylated derivatives of Crystal Violet.

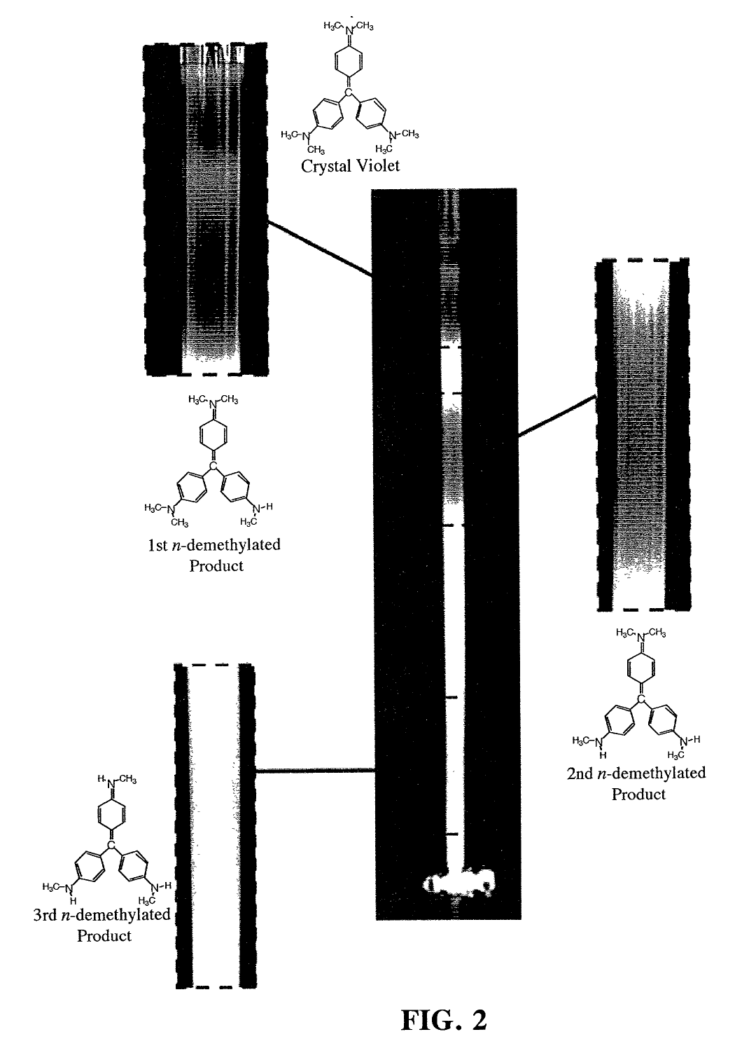

### DETAILED DESCRIPTION OF THE INVENTION

Extensively conjugated cationic molecules with appropriate structural features will accumulate into the mitochondria of living cells, a phenomenon typically more prominent in tumor cells than in normal cells. It has now been found that a variety of tumor cells also retain pertinent cationic structures for longer periods of time compared to normal cells. While not being bound to a particular mode of action, the present method utilizes mitochondrial targeting as a selective therapeutic strategy of relevance for both chemotherapy and photochemotherapy of neoplastic diseases in general, and leukemia in particular.

The present invention is directed to the use of triarylmethane (TAM⁺) dyes, the preferred dye being MV2B, for photochemotherapy of neoplastic conditions. MV2B and the related compounds disclosed herein stain neoplastic cell mitochondria with efficiency and selectivity. Upon exposure to suitable wavelengths of energy, the dyes exhibit pronounced and selective phototoxicity toward neoplastic cells. As illustrated in the Examples that follow, MV2B exhibits pronounced phototoxicity toward L1210 leukemia cells but comparatively small toxic effects toward normal hematopoietic cells (murine granulocyte-macrophage progenitors, CFU-GM cells). On the basis of a comparative examination of chemical, photochemical, and phototoxic properties of MV2B and other triarylmethane dyes, certain interdependencies between molecular structure and selective phototoxicity toward tumor cells have been identified. These structure-activity relationships provide useful guidelines for the novel purging protocols described herein, protocols that selectively eliminate residual tumor cells from autologous bone marrow grafts with minimum toxicity to normal hematopoietic stem cells.

General Synthetic Approach:

Compounds according to the present invention can be synthesized using two approaches. The halogenated compounds are prepared by treating the parent compound with molecular bromine. See Kobayashi et al. (1977) J. Chem. Res. (S) 215. The TAM+ dye is dissolved in glacial acetic acid and a solution of bromine, also in glacial acetic acid, is slowly added thereto. The dye solution is constantly stirred at controlled temperature and under a positive pressure of nitrogen during the addition of the bromine. The stoichiometry of the final products is controlled by the initial concentration of reactants, temperature, and reaction time.

More rigid analogs are prepared via the covalent linking of two TAM⁺ aromatic rings at the ortho position. The synthesis of these analogs follows a synthetic route developed by Davis, see U.S. Pat. No. 3,344,189, incorporated herein. Briefly, a suitable amount of the parent dye is dissolved in 85% sulfuric acid and the reaction mixture is slowly heated and kept at 205° C. for about 30 minutes. After the reaction is completed, the reaction mixture is cooled, poured onto ice, and the acid partially neutralized with aqueous ammonia. Sodium dithionite is subsequently added to reduce the dye to its respective leuco form. In this step, the acid remaining in solution is neutralized. The precipitate formed in this step is filtered, washed with water, and dried. This intermediate (leuco) compound is purified by recrystallization from toluene, and subsequently oxidized to the respective carbinol base by treatment with lead peroxide in a mixture of glacial acetic acid and sulfuric acid. After four hours at room temperature, the lead sulfate is filtered off, and the carbinol base precipitated with sodium hydroxide. After recrystallization of the carbinol base, this last intermediate is finally converted to the chloride salt of the product with diluted hydrochloric acid.

The following experiments are provided for illustrative purposes only, in an effort to describe the claimed invention clearly and completely. It is understood that the experiments described below do not limit the invention claimed herein in any fashion.

Chemicals:

Chlorine salts of the triarylmethane dyes ethyl violet (EV⁺), victoria blue R (VBR⁺), victoria pure blue BO (VPBBO⁺) from Aldrich Chemical (Milwaukee, Wis.), and CV⁺ from Sigma (St. Louis, Mo.) were recrystallized from methanol and dried under vacuum. The purity of recrystallized TAM⁺ dyes was assessed by thin-layer chromatography (TLC, silica gel, methanol-acetic acid 95:5, vol/vol). N-2-hydroxyethyl piperazine-N′-2-ethanesulfonic acid (HEPES) was obtained from Research Organics (Cleveland, Ohio); methylcellulose (4000 cPs) from Fluka Chemical; recombinant granulocyte/macrophage colony stimulating factor (GM-CSF; murine sequence) from R&D Systems (Minneapolis, Minn.); bovine serum albumin (BSA), fetal bovine serum (FBS), and alpha-modified Dulbecco's medium (alpha-medium) from Sigma; and minimal essential media and newborn calf serum (NCS) from Gibco BRL. Rhodamine 123 (Rh123) from Molecular Probes and 1-octanol from Aldrich were used as supplied. Water was distilled, deionized, and filtered before use (Millipore Milli-Q system; resistivity, 18 MΩ cm). The characterization of reaction photoproducts was carried out by electronic spectroscopy and TLC as so described elsewhere.¹⁷

Spectroscopic and Photochemical Investigations in Solution:

Spectrophotometric studies were performed with a Shimadzu UV-2101PC spectrophotometer. 1-Octanol/water partition coefficients¹⁸ (P), were determined at 25° C. using equal volumes of water and 1-octanol. Typically, five distinct solutions of each dye in the concentration range between 1 μM and 10 μM were prepared in deionized water and subsequently equilibrated with 1-octanol. After the equilibrium was reached, the final dye concentrations in the aqueous and/or organic phases were determined by absorption spectroscopy. For the measurement of photobleaching quantum efficiencies, the samples were placed in standard 10-mm (optical path) quartz cells at a distance of approximately 10 cm from the light source and photolyzed using the 532-nm line of an Nd:YAG laser model 7010 from Continuum operating at a repetition rate of 10 Hz. The defocused laser beam (circular profile with a diameter of about 5 mm) was directed to the center of the quartz cell. The absolute photolysis energy (or the number of 532-nm photons per laser pulse) was kept constant over the course of any specific experiment. The temperature-controlled cell holder allowed continuous magnetic stirring of the samples during photolysis. The photobleaching quantum efficiencies were based on the photobleaching efficiency of a classical chemical actinometer, potassium ferrioxalate,^{19,20} and determined considering only the first 5 to 10% decrease in dye concentration. The photolysis energy was adjusted to appropriate levels with the use of a calibrated solid-state joulemeter model PM30VI from Molectron.

The laser flash photolysis equipment used in the Examples is similar to a previously-described system.²¹ The major assembly components are a nanosecond dye laser (Continuum, ND6000) pumped by an Nd:YAG laser (Continuum, 7010), used as the excitation light source; a 300 W xenon arc lamp system (Oriel, 66084), which provides the analysis beam; a monochromator (CVI, CM110); a red-sensitive photomultiplier tube (Hamamatsu, R446); and a dual-channel 600-MHz digital oscilloscope (LeCroy, 9360).

Cells:

L1210 murine leukemia cells (CCL 219; American Type Culture Collection, Manassas, Va.) were cultured in alpha-medium supplemented with 10% FBS, incubated in a humidified atmosphere of 5% CO₂ in air and harvested in exponential growth phase. Rat basophilic leukemia (RBL) cells were plated at a density of 2.3×10⁵ cells per 2 mL of growth media (minimal essential media (GibcoBRL) supplemented with 10% FBS and 10% NCS) on observation dishes with a glass coverslip bottom and allowed to adhere overnight at 37° C. Female C57BL/6J×DBA/2J mice (approximately 6 months old; The Jackson Laboratory, Bar Harbor, Me.) served as a source of normal bone marrow cells.

Two-Photon Microscopy:

RBL cells were incubated in the dark at a concentration of 1.1×10⁵ cells/mL for periods ranging from 1 minute to 1 hour with fresh growth media containing 0.2 μM CV⁺ or EV⁺ and subsequently incubated for 30 minutes in the dark with fresh growth media containing 0.05 μM Rh123. After a final cycle of washing and incubation for 15 minutes in fresh growth media, the media were replaced by modified Tyrode's solution containing 5 mM glucose and bovine serum albumin, and the cells were subjected to two-photon imaging. The examination of TAM⁺ accumulation into mitochondria was carried out through the comparison of the spatial distribution of the fluorescence of these dyes in the cellular environment with that of Rh123 (a classical mitochondrial marker¹⁻³) and the endogenous NAD(P)H. Fluorescence distributions within living RBL cells were obtained with a laser scanning multiphoton microscope (Bio-Rad MP1024). Pulsed (75 fs, 80 Mhz) infrared 750-nm laser light was focused to a diffraction-limited spot with a Zeiss F-Fluar oil immersion objective (NA=1.3) and raster-scanned across the sample. Three external detectors located in the Fourier plane of the microscope and equipped with appropriate sets of longpass dichroic and bandpass filters (from Chroma) were used for the simultaneous detection of two-photon excited fluorescence of NAD(P)H (400-500 nm), Rh123 (520-550 nm), and CV⁺ or EV⁺ (600-660 nm). To obtain sufficient fluorescence from CV⁺, EV⁺, and cellular NAD(P)H autofluorescence (all inefficient fluorophores), average excitation powers of approximately 18 mW were required. A detailed description of the experimental setting used in two-photon microscopy is known in the art.²²

Uptake of Crystal Violet by L1210 Cells:

L1210 cells were suspended at a concentration of 1.0×10⁶ cells/mL in HEPES-buffered (10 mM, pH 7.4) alpha-medium supplemented with FBS (12%), and the TAM⁺ dyes were added from 1.0×10⁻⁴ M stock solutions in 50% ethanol to final concentrations of 1.0×10⁻⁶ M. The cells were subsequently incubated in the dark for 1 hour at 37° C., pelleted, washed with dye-free medium, and extracted with ethanol (1 mL per 5×10⁻⁶ cells). The dye content of each extract was determined spectrophotometrically using calibration curves assembled using suitable dilutions of authentic dye in ethanol extracts of cells that had been incubated in dye-free medium Control experiments demonstrated that under these experimental conditions (1.0×10⁻⁶ cells/mL, 12% FBS) and within the time frames when the measurements were taken, TAM⁺ dyes at a final concentration of 1.0×10⁻⁶ M showed negligible or no dark toxicity toward L1210 cells.

Dye-Sensitized Photoinactivation of Cells:

The cells were incubated for 60 minutes in the presence of the TAM⁺ dyes as described above, washed once with dye-free HEPES-buffered buffered alpha-medium supplemented with 5% FBS, and resuspended at a density of 10⁶ cells/mL in HEPES-buffered alpha-medium supplemented with 12% FBS. Capped clear polystyrene tubes (15 mL) containing the cell suspension (2 mL) were mounted on a Plexiglas disk that rotated at approximately 60 rpm between two banks of tubular fluorescent lights (five lights per bank; F20T12.CW; General Electric, Cleveland, Ohio)²³ and irradiated for up to 90 minutes. The fluence rate at the sample site was 27 W/m², as determined by a model S351A power meter (United Detector Technology, Hawthorne, Calif.) equipped with a model 262 detector and radiometric filter number 1158. For select experiments, the cell suspensions were treated with oxygen or argon immediately before the photoirradiation period as previously described.²⁴ After irradiation, the cells were washed twice with dye-free HEPES-buffered alpha-medium supplemented with 5% FBS, and all subsequent manipulations were performed in the dark or under low level ambient light.

Chemical Synthesis and Isolation:

Compounds according to the present invention can be synthesized via enzymatic N-dealkylation of the next-higher homology in the series. See Gadelha et al. (1992) Chem-Biol. Interactions, 85:35-48, incorporated herein by reference. Specifically, starting with the parent per-alkylated compound, alkyl groups can be sequentially removed using horseradish peroxidase. Because the reaction requires H₂O₂ to proceed, the extent of dealkylation is controlled by limiting the initial concentration of H₂O₂ and allowing the reaction to go to completion. Therefore, the experimental conditions (given in Gadelha et al., supra) can be tailored to maximize the yield of each product of interest, thereby greatly facilitating the isolation of the product via HPLC.

The desired product is isolated via reverse-phase HPLC, using an isocratic elution profile, 90:10 acetonitrile:50 mM aqueous HClO_{4.}

In addition to HPLC, larger quantities of the disclosed compounds can be isolated and/or concentrated using reduced-pressure column chromatography. Here, silica gel is washed with an aqueous salt solution of from about 0.5 to 5.0 M. Sodium chloride or other salts can be used. The washed silica is subsequently filtered to remove the excess salt solution. The pre-treated, water-deactivated silica gel is made into a slurry in 2-propanol and packed into columns under reduced pressure. The dye mixtures to be separated are dissolved in 2-propanol and the column is run using 2-propanol as the mobile phase.

In Vitro Clonal Assays:

The survival of photoinactivated and untreated L1210 leukemia cells was assessed using an in vitro clonal assay as described elsewhere.²⁵ CFU-GM cells were assayed as previously described.^{26,27} Cells that had been exposed to dye but not to light, to light but not to dye, or to neither dye nor light, served as controls.

Intracellular Distribution of CV⁺ and EV⁺ in Living Cells:

Two-photon laser scanning microscopy^{22,28} was used to obtain information on intracellular distribution and mitochondrial accumulation of Crystal Violet (CV⁺) and Ethyl Violet (EV⁺) in RBL cells. The experimental strategy used a mitochondrial marker (Rh123) and cellular NAD(P)H autofluorescence²⁹ to probe the cellular distribution of TAM⁺ dyes and to characterize early alterations in mitochondria structure and bioenergetics associated with the cytotoxic effect of these compounds. Simultaneous two-photon images of NAD(P)H, Rh123, and TAM⁺ fluorescence were obtained by exciting RBL cells at 750 nm with a Ti:Sapphire laser (75 fs, 80 MHz, 18 mW) as the excitation source. No morphologic alterations or induction of fluorescence attributed to cellular photodamage was observed during the brief imaging period (approximately 3 seconds), and the bleaching of the three fluorophores was also negligible during this period. The cell fluorescence was analyzed in three detection channels for the simultaneous characterization of the spatial distribution of each fluorophore of interest, NAD(P)H, Rh123, and TAM⁺, inside the RBL cells.

The images obtained in the control experiments without TAM⁺ and Rh123 displayed bright punctate NAD(P)H autofluorescence distribution. This distribution was analogous to the distribution observed for mitochondria stained only with Rh123, but with the addition of a background autofluorescence distribution throughout the cytoplasm and to a lesser degree in the nucleus. In the case of cells incubated for 10 minutes in the presence of CV⁺ and subsequently with Rh123, the fluorescence associated with CV⁺, Rh123, and NAD(P)H had similar perinuclear distributions in the cell, which is consistent with mitochondrial localization^{1,29}. Rh123 is known to stain mitochondria with remarkable efficiency and selectivity. For this particular dye, the membrane potential-driven contribution of the mitochondrial accumulation phenomenon is clearly the dominant contribution. Accordingly, on depolarization of the mitochondrial membrane of living cells, Rh123 no longer accumulates or is retained in the mitochondria.^{1,2} After 1 hour of CV⁺ incubation, Rh123 was no longer efficiently retained by RBL cell mitochondria, the mitochondria appeared swollen (approximately 2-3 μm in diameter), and cellular autofluorescence was less intense and less punctate. This observation indicates that the mitochondrial inner membrane potential has been reduced and NADH has been oxidized to NAD⁺. The approach of monitoring NAD(P)H fluorescence in living cells for the assessment of mitochondrial bioenergetics was originally introduced by Chance.²⁹ In model studies carried out with isolated rat liver mitochondria, the depolarization of the mitochondrial membrane by CV⁺ has been attributed to uncoupling³⁰ and induction of mitochondrial permeability transition.³¹

When EV⁺ was used in place of CV⁺, similar mitochondrial effects were observed. The comparison of the fluorescence patterns of EV⁺, Rh123, and NAD(P)H after 10 minutes of RBL incubation with EV⁺ indicated that this dye also accumulates into cell mitochondria. However, significant EV⁺ fluorescence background was observed throughout the cytoplasm, suggesting that a substantial fraction of the EV⁺ molecules taken up by RBL cells may also localize in other subcellular compartments. In addition, after depolarization of the mitochondrial membrane and release of Rh123, substantial EV⁺ fluorescence was still observed in mitochondrial regions.

For extensively conjugated cationic structures displaying appropriate lipophilic/hydrophilic character, the contribution of membrane partitioning on the mechanism of dye accumulation into the cytosol and inside cell mitochondria may be negligible. In these cases, mitochondrial accumulation appears to be controlled primarily by membrane potential-driven electrophoresis and chemical potentials, as described by the Nernst equation.² On increasing the lipophilic character of the conjugated cationic structure, a higher contribution from the partitioning phenomena is expected to occur, and consequently mitochondrial accumulation may be preserved even after the mitochondrial membrane is depolarized. For highly lipophilic compounds, membrane partitioning can be expected to represent the dominant contribution. A higher contribution of membrane partitioning phenomena on the mechanism of mitochondrial accumulation of EV⁺, compared with CV⁺, was indicated by the fact that substantial EV⁺ fluorescence was still observed in mitochondrial regions after depolarization of the mitochondrial membrane.

Phototoxic Effects of TAM⁺ Dyes Toward Leukemia and Normal Hematopoietic Cells:

To explore whether CV⁺ and MV2B can promote the selective destruction of tumor cells with minimum toxicity toward normal cells, a model preclinical study was conducted in which the phototoxicity of CV⁺ and MV2B dyes toward leukemia (L1210) and normal hematopoietic (murine CFU-GM) cells was compared under experimental conditions in which the respective thermal (dark) toxicity was small for both cell lines (1.0×10⁻⁶ M dye, 1.0×10⁶ cells/mL; 12% FBS). The choice of murine CFU-GM cells to assess the toxicity to normal hematopoietic cells was motivated by the fact that they are relatively frequent in bone marrow (thus allowing depletions over two to three orders of magnitude to be documented) and also because, in purging applications, a good correlation is often found between the preservation of CFU-GM cells and the recovery of the neutrophil compartment. For certain purging agents, the survival of CFU-GM cells has also proven to be a reliable indicator of the radioprotective capacity of purged marrow.²⁶

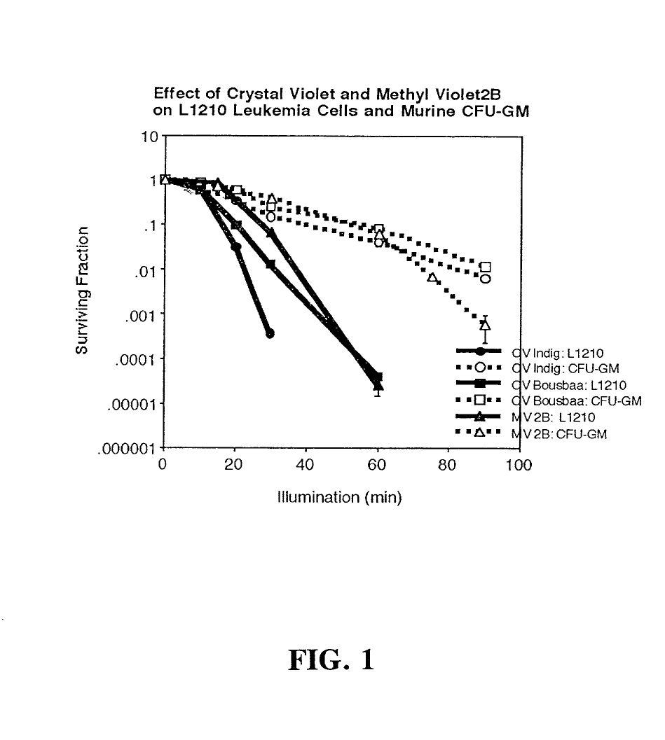

FIG. 1 shows the efficiency of CV⁺ and MV2B to photoinactivate L1210 leukemia cells selectively as a function of time of light exposure. The comparison between the data shown in the solid-line traces (L1210) and the broken-line traces (CFU-GM) of FIG. 1 clearly indicates that both CV⁺ and MV2B have utility for photodynamic therapy due to their ability to destroy cancer cells selectively. After relatively short periods of light exposure (c.a. 20 minutes), the surviving fractions of L1210 cells represented only 0.3 to 0.4% of their initial values, whereas in the case of CFU-GM cells the respective surviving fractions were still in the range of 60 to 70% of their initial values.

Photoreactivity Versus Observed Toxicity:

The extent to which a specific TAM⁺ dye is capable of inducing specific destruction of residual tumor cells in bone marrow grafts depends primarily on its preferential uptake by tumor cells compared with normal cells, the site(s) of subcellular localization, and the quantum efficiency of the photochemical events that lead to the lethal toxic effects. TAM⁺ dyes show very short singlet lifetimes in low viscosity media because of fast, non-radiative relaxation processes that occur via rotational motions of their aromatic rings.^{17,35,36} When free in aqueous media, the photoreactivity of TAM⁺ dyes is extremely poor, and no significant phototoxicity should be expected from these dyes under such circumstances. However, in aqueous media, TAM⁺ dyes bind efficiently to a variety of biopolymer polyelectrolytes through noncovalent interactions, including proteins and nucleic acids.^{15,17,37}

Accordingly, in complex biologic systems, these photosensitizers are not expected to be found free in solution to any significant extent, but rather bound to biopolymers and supramolecular structures. When TAM⁺ molecules are located in binding micro-environments that render steric hindrance to the rotational motions of their aromatic rings, the efficiency of non-radiative relaxation processes decreases compared with dye molecules free in aqueous media. As a result, fluorescence and intersystem crossing become more competitive events, and photoreactivity tends to increase.^{17,37}

To explore how the photoreactivity of the TAM⁺ dyes in complex biologic environments correlates with the observed phototoxic effects toward tumor and normal cells, the photo-bleaching efficiency of TAM⁺ dyes noncovalently bound to a model biologic host (bovine serum albumin, BSA) was measured. For the case of CFU-GM cells, the efficacy of photo-induced cell destruction mediated by TAM⁺ dyes precisely paralleled the photochemical reactivity of these photosensitizers. Both, phototoxicity and photoreactivity (Table 1) followed the decreasing order EV⁺>VPBBO⁺>CV⁺>VBR⁺. However, the toxic effect of CV⁺ toward L1210 leukemia cells was substantially higher than what would be expected solely on the basis of photochemical considerations. In fact, the phototoxicity of CV⁺ toward L1210 cells was comparable to that observed for the case of the most photoreactive triarylmethane tested, EV⁺.

Phototoxicity Versus Cellular Uptake:

Data on cellular uptake (Table 1) for the dyes tested herein indicated that CV⁺ is the dye most efficiently taken up by L1210 cells. Thus, enhanced tumor cell uptake is thought to be a major mechanistic mode of action resulting in observed behavior of CV⁺, and provides a plausible explanation for the fact that the phototoxic effect of CV⁺ toward L1210 cells does not follow the trend predicted by the relative photoreactivity along the TAM⁺ dye series. For the other TAM⁺ dyes tested to date, EV⁺, VPBBO+, and VBR+, both the efficiency of cellular uptake by L1210 cells and dye photoreactivity paralleled phototoxicity.

TABLE 1
Photobleaching Efficiencies, Partition Coefficients (P),
and Cellular Uptake Values for TAM⁺ Dyes

| | | | Dye Uptake by |
|---|---|---|---|
| | Photobleaching | | L1210 Cells |
| | Efficiency^{a} | | (x 10⁻⁷ |
| | (x 10⁵) | (P) | Molecules/Cell) |
| EV⁺ | 3.3 | 237 | 17.6 |
| VPBBO+ | 3.0 | 180 | 11.6 |
| CV⁺ | 1.3 | 2.4 | 21.4 |
| VBR+ | 0.5 | 39 | 8.8 |

^{a}10 mM buffer pH 7.3. {Dye} = 10 μM; {BSA} = 40 μM

Lipophilic/Hydrophilic Character and Cellular Uptake:

With the exception of CV⁺, the relative efficiency by which L1210 cells take up TAM⁺ molecules correlates with the lipophilic/hydrophilic character of these compounds, as assessed through the measurement of 1-octanol/water partition coefficients (Table 1). For VBR+, VPBBO+, and EV⁺, the higher the partition coefficient, the higher the cellular accumulation. However, the dye showing the lowest 1-octanol/water partition coefficient (P) among the TAM⁺ dyes considered herein, CV⁺ (P=2.4), is the dye most efficiently taken up by L1210 cells. The enhanced L1210 cellular uptake observed for the case of CV⁺, compared with the other more lipophilic TAM⁺ structures, is in keeping with the hypothesis that the cellular uptake and mitochondrial accumulation and retention of a cationic compound displaying appropriate structural features can be primarily driven by membrane potentials rather than by the partitioning phenomena. In the case of the model mitochondrial dye Rh123, whose cellular uptake and mitochondrial accumulation is known to be driven almost exclusively by membrane potentials,^{1,2} the 1 -octanol/water partition coefficient is only 0.24 as measured under exactly the same conditions as those used for the TAM⁺ dyes. Therefore, because the values of partition coefficients of CV⁺ and Rh123 are relatively close, it is reasonable to presume that the enhanced CV⁺ uptake by L1210 cells is a direct consequence of a more appropriate lipophilic/hydrophilic character of this dye, as compared to the other TAM⁺ structures considered here. The high values of partition coefficients of VBR+(39), VPBBO+(180), and EV⁺ (237) suggest that for these dyes, the membrane partitioning phenomena must represent a much more pronounced contribution to the respective mechanisms of subcellular distribution and mitochondrial accumulation. Indeed, the two-photon laser microscopy data suggested that mitochondrial membrane partitioning plays a more prominent role in the mitochondrial localization and accumulation of EV⁺ than it does for CV⁺.

### BIBLIOGRAPHY

1. Chen L B. 1988. Mitochondrial membrane potential in living cells. Ann Rev Cell Biol 4:155-181.

2. Davis S, Weiss M J, Wong J R, Lampidis T J, Chen L B. 1985. Mitochondrial and plasma membrane potentials cause unusual accumulation and retention of rhodamine 123 by human breast adenocarcinoma-derived MCF-7 cells. J Biol Chem 260:13844-13850.

3. Kawakami M, Koya K, Ukai T, Tatsuta N, Ikegawa A, Ogawa K, Shishido T, Chen L B. 1997. Synthesis and evaluation of novel rhodacyanine dyes that exhibit antitumor activity. J Med Chem 40:3151-3160.

4. Modica-Napolitano J S, Brunelli B T, Keizo K, Chen L B. 1998. Photoactivation enhances the mitochondrial toxicity of the cationic rhodacyanine MKT-077. Cancer Res 58:71-75.

5. Koya K, Li Y, Wang H, Ukai T, Tatsuta N, Kawakami M, Shishido T, Chen L B. 1996. MKT-077, a novel rhodacyanine dye in clinical trials, exhibits anticarcinoma activity in preclinical studies based on selective mitochondrial accumulation. Cancer Res 56:538-543.

6. Ara G, Apprille J R. Malis C D, Kane S B, Cincotta L, Foley J, Bonventre J V, Oseroff A R. 1987. Mechanism of mitochondrial photosensitization by the cationic dye, N,N-bis(2-ethyl-1,3-dioxolane)krypto-cyanine (EDKC): preferential inactivation of complex I in the electron transport chain. Cancer Res 47:6580-6585.

7. Morgan J, Whitaker J E, Oseroff A R. 1998. GRP78 induction by calcium ionophore potentiates PDT using the mitochondrial targeting dye Victoria Blue BO. Photochem Photobiol 67:155-164.

8. Nussenzweig V, Sonntag R, Biancalana A, Freitas J L P, Amato Neto V, Kloetzel J. 1953. Action of certain dyes on T. cruzi in vitro. The use of gentian violet to prevent the transmission of Chagas. Hospital (Rio J) 44:731-744.

9. Docampo R, Moreno S N J, Muniz R P A, Mason R P. 1983. Light enhanced free radical formation and trypanocidal action of gentian violet (crystal violet). Science 220:1292-1294.

10. Gadelha F R, Moreno S N J, De Souza W, Cruz F S, Docampo R. 1989. The mitochondrion of Tiypanosoma cruzi is a target of CV toxicity. Molec Biochem Parasitol 34:117-126.

11. Reszka K, Cruz F S, Docampo R. 1986. Photosensitization by the trypanocidal agent crystal violet. Type I versus type II reactions. Chem Biol Interactions 58:161-172.

12. Docampo R, Moreno S N J, Gadeiha F R, De Souza W, Cruz F. 1988. Prevention of Chagas' disease resulting from blood transfusion by treatment of blood: toxicity and mode of action of gentian violet. Biomed Environ Sci 1:406-413.

13. Ramirez L E, Lages-Silva E, Pianetti G M, Rab˜lo R M C, Bordin J O, Mornes-Souza H. 1995. Prevention of transfusion-associated Chagas' disease by sterilization of Trypanosoma cruzi-infected blood with gentian violet, ascorbic acid, and light. Transfiusion 35:226-230.

14. Moraes-Souza H, Bordin J O. 1996. Strategies for prevention of transfusion-associated Chagas' disease. Transf Med Rev 10: 161-170.

15. Duxbury D F. 1993. The photochemistry and photophysics of triphenylmethane dyes in solid and liquid media. Chem Rev 93:381-433.

16. Fisher V, Harrelson W G Jr, Chingel C F, Mason R P. 1984. Spectroscopic studies of cutaneous photosensitizing agents. V. Spin trapping and direct electron spin resonance investigations of the photoreduction of gentian (crystal) violet. Photochem Photobiol 7:11-119.

17. Baptista M S, Indig G L. 1998. Effect of BSA binding on photophysical and photochemical properties of triarylmethane dyes. J Phys Chem 102B:4678-4688.

18. Leo A, Hansch C, Elkins D. 1971. Partition coefficients and their uses. Chem Rev 71:525-016.

19. Hatchard C G, Parker C A. 1956. A new sensitive chemical actinometer II. Potassium ferrioxalate as a standard chemical actinometer. Proc R Soc London, Ser A 235:518-536.

20. Hamai 5, Hirayama F. 1983. Actinometric determination of absolute fluorescence quantum yields. J Phys Chem 87:83-89.

21. Liao Y, Bohne C. 1996. Alcohol effect on equilibrium constants and dissociation dynamics of xanthone-cyclodextrin complexes. J Phys Chem 100:734-743.

22. Albota M A, Xu C, Webb W W. 1998: Two-photon fluorescence excitation cross section of biomolecular probes from 690 nm to 960 nm. Appl Optics 37:7352-7356.

23. Anderson G S, Guenther W H H, Searle li, Bilitz J M, Krieg M, Sieber F. 1996. Inactivation of photosensitizing merocyanine dyes by plasma, serum and serum components. Photochem Photobiol 64:683-687.

24. Gaffney D K, Schober S L, Sieber F. 1990. Merocyanine 540-sensitized photoinactivation of leukemia cells: Role of oxygen and effects on plasma membrane integrity and mitochondrial respiration. Exp Hematol 18:23-26.

25. Sieber F, Spivak J L, Sutcliffe A M. 1984. Selective killing of leukemic cells by merocyanine 540-mediated photosensitization. Proc Natl Acad Sci USA 81:7584-7587.

26. Yamazaki T, Sato Y, Sieber F. 1997. Role of cytoprotective mechanisms in the photochemical purging of autologous bone marrow grafts. Exp Hematel 25:629-637.

27. Iscove N N, Sieber F, Winterhalter K H. 1974. Erythroid colony formation in cultures of mouse and human bone marrow: analysis of the requirement for erythropoietin by gel filtration and affinity chromatography on agarose-concanavalin-A. J Cell Physiol 83:309-320.

28. Denk W, Strickler J H, Webb W W. 1990. 2-photon laser scanning fluorescence spectroscopy. Science 248:73-76.

29. Chance B. 1970. Fluorescent probe environment and the structural and charge changes in energy coupling of mitochondrial membranes. Proc Natl Acad Sci USA 67:560-564.

30. Moreno S N J, Gadelha F R, Docampo R. 1988. Crystal violet as an uncoupler of oxidative phosphorylation in rat liver mitochondria. J Biol Chem 263: 12493-12499.

31. Kowaltowski A J, Turin J, Indig G L, Vercesi A E. 1999. Mitochondrial effects of triarylmethane dyes. J Bioenerg Biomembr 31:579-588.

32. Fiedorowicz M, Pituch-Noworolska A, Zembala M. 1997. The effect of victoria blue BO on peripheral blood mononuclear and leukemic cells. Photochem Photobiol 65:855-861.

33. Viola A, Hadjur C, Jeunet A, Julliard M. 1996. Electron paramagnetic resonance evidence of the generation of superoxide and hydroxyl radicals by irradiation of a new photodynamic therapy photo-sensitizer, Victoria Blue BO. J Photechem Photobiol B: Biol 32:49-58.

34. Docampo R, Moreno S N J, Cruz F S. 1988. Enhancement of the cytotoxicity of crystal violet against Trypanospoma cruzi in the blood by ascorbate. Molec Biochem Parasitol 27:241-248.

35. Sundstrom V, Gilibro T, Bergstrom H. 1982. Picosecond kinetics of radiationless relaxations of triphenylmethane dyes. Evidence for a rapid excited-state equilibrium between states of differing geometry. Chem Phys 73:439-458.

36. Vogel M, Rettig W. 1985. Efficient intramolecular fluorescence quenching in triphenylmethane dyes involving excited states with charge separation and twisted conformations. Ber Bunsen-Ges Phys Chem 89:962-968.

37. Bartlett J A, Indig G L. 1999. Effect of self-association and protein binding on the photochemical reactivity of triarylmethanes. Implications of non-covalent interactions on the competition between photosensitization mechanisms type I and type II. Photochem Photobiol 70:490-498.

38. Foote C S. 1968. Mechanism of photosensitized oxidation. Science 162:963-970.

39. Foote C S. 1991. Definition of type I and type II photosensitized oxidation. Photochem Photobiol 54:659.

40. Kraljic I, El Moshni S. 1978. A new method for the detection of singlet oxygen in aqueous solution. Photochem Photobiol 28:577-581.

41. Lueck H B, Rice B L, McHale J L. 1992. Aggregation of triphenylmethane dyes in aqueous solution: dimerization and trimerization of crystal violet and ethyl violet. Spectrochim Acta 48A, 819-828.

42. Kasha M, Rawls H R, El-Bayoumi M A. 1965. The exciton model in molecular spectroscopy. Pure Appl Chem 11:371-392.

43. Jockusch S, Turro N J. 1999. Radical addition rate constants to acrylates and oxygen: a-hydroxy and a-amino radicals produced by photolysis of photoinitiators. J Am Chem Soc 121:3921-3925.

44. Chen, L. B. (1998) Mitochondrial Membrane Potential in Living Cells. Ann. Rev. Cell Biol. 4:155-181.

45. Yamazaki, T. and Sieber, F. (1997) The Alkyl-lysophospholipid, ET-18-OCH₃ Synergistically Enhances the Merocyanine 540-mediated Photoinactivation of Leukemia Cells: Implications for the Extracorporeal Purging of Autologous Hematopoietic Stem Cells, Bone Marrow Transplantation, 19:113-119.

46. Yamazaki, T., et al. (1997) Role of Cytoprotective Mechanisms in the Photchemical Purging of Autologous Bone Marrow Grafts, Experimental Hematology 25:629-637.

What is claimed is:

1. A method of purging malignant cells from a mixture containing malignant and non-malignant cells, the method comprising:
(a) contacting the mixture with a compound selected from the group consisting of: 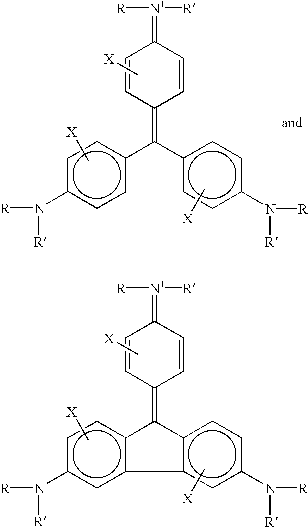
wherein each R and R′ is independently selected from the group consisting of hydrogen and C₁-C₆ linear or branched alkyl, and each X is independently selected from the group consisting of hydrogen, chloro, fluoro, bromo, and iodo;
(b) exposing the mixture from step (a) to radiation of a suitable wavelength to photoactivate the compound, thereby inducing death of malignant cells in the mixture.

2. The method of claim 1, wherein in step (a), the mixture is contacted with a compound wherein five of the R and R′ substituents are methyl, and one of the R or R′ substituents is hydrogen.

3. The method of claim 1, wherein the mixture comprises bone marrow cells.

4. The method of claim 3, wherein the bone marrow cells are cells taken from a patient suffering from leukemia, disseminated multiple myeloma, or lymphoma.

5. The method of claim 3, wherein the bone marrow cells are human bone marrow cells.

6. A method of killing cancer cells or inhibiting growth of cancer cells, in vitro, in vivo, or ex vivo, the method comprising:
(a) contacting the cancer cells with a compound selected from the group consisting of: 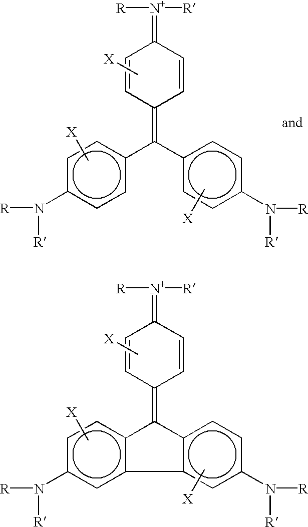
wherein each R and R′ is independently selected from the group consisting of hydrogen and C₁-C₆ linear or branched alkyl, and each X is independently selected from the group consisting of hydrogen, chloro, fluoro, bromo, and iodo;
(b) exposing the cancer cells from step (a) to radiation of a suitable wavelength to photoactivate the compound, whereby cancer cell death or cancer cell growth inhibition results.

7. The method of claim 6, wherein in step (a), the cancer cells are contacted with the compound in vitro.

8. The method of claim 6, wherein in step (a), the cancer cells are contacted with the compound in vivo.

9. The method of claim 6, wherein in step (a), the cancer cells are contacted with the compound ex vivo.

10. The method of claim 6, wherein in step (a), the cancer cells are contacted with a compound wherein five of the R and R′ substituents are methyl, and one of the R or R′ substituents is hydrogen.

11. A pharmaceutical composition for the photodynamic purging malignant cells from a mixture containing malignant and non-malignant cells, the composition comprising: an amount of a compound selected from the group consisting of: 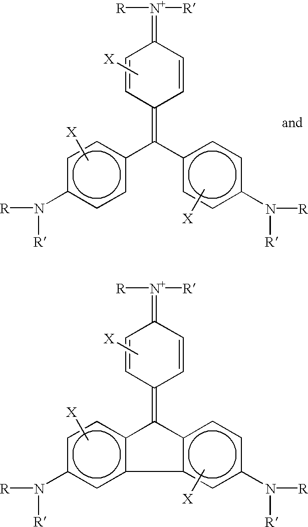
wherein each R and R′ is independently selected from the group consisting of hydrogen and C₁-C₆ linear or branched alkyl, and each X is independently selected from the group consisting of hydrogen, chloro, fluoro, bromo, and iodo, and pharmaceutically-suitable salts thereof, in combination with a pharmaceutically-suitable carrier, the amount being effective to kill malignant cells when exposed to radiation that activates the compound.

12. A method of inactivating alloreactive cells, including T cells, in a mixture containing alloreactive and non-alloreactive cells, the method comprising:
(a) contacting the mixture with a compound selected from the group consisting of: 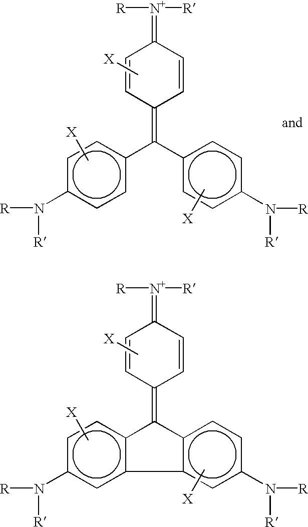
wherein each R and R′ is independently selected from the group consisting of hydrogen and C₁-C₆ linear or branched alkyl, and each X is independently selected from the group consisting of hydrogen, chloro, fluoro, bromo, and iodo;
(b) exposing the mixture from step (a) to radiation of a suitable wavelength to photoactivate the compound, thereby inducing inactivation of alloreactive cells in the mixture.
